(19) United States
(12) Patent Application Publication
(10) Pub. No.: US 2022/0249269 A1
(43) Pub. Date: Aug. 11, 2022

(54) ADAPTABLE FINGER SPLINT

(71) Applicant: Jared Sharnowski, Novi, MI (US)

(72) Inventor: Jared Sharnowski, Novi, MI (US)

(21) Appl. No.: 17/171,435

(22) Filed: Feb. 9, 2021

(51) Int. Cl. A61F 5/058

(52) U.S. Cl. CPC A61F 5/05875

(57) ABSTRACT

A number of variations may include a finger splint including a plurality of tubular components arranged along a shared axis of rotation. The tubular components may be connected by at least one connective portion. The tubular components may also include, individually, a hook and loop patch disposed on an outer portion of the tubular component. A hook and loop strap may be constructed and arranged for providing tension across the tubular components such that a wearers finger may be immobilized within the finger splint.

### TECHNICAL FIELD

The field to which the disclosure generally relates includes medical splints.

### BACKGROUND

Finger splints allow for the securing of injured finger and prevention of movement such that the finger may heal. Splints disable the patient from using their finger and associated hand resulting in less productivity of the patient. To overcome this, patients may remove their finger splint and risk re-injuring their healing finger and losing their finger splint.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

An adjustable finger splint may include a plurality of hollow tubes that each may include a respective inner surface opposite an outer surface; at least one hook and loop patch disposed on the outer surface; and at least one hook and loop strap constructed and arranged to mechanically connect to the at least one hook and loop patch such that the plurality of hollow tubes are at least semi-rigidly aligned approximately on the same center axis to provide for immobilization of a wearer's finger.

An adjustable finger splint may include a first hollow tube that may include a first inner surface opposite a first outer surface and defining a first break in the circumference of the first tube; a second hollow tube that may include a second inner surface opposite a second outer surface and defining a second break in the circumference of the second tube; at least one connective portion mechanically joining the first tube to the second tube; a first hook and loop patch disposed on the first outer surface; a second hook and loop patch disposed on the second outer surface; and a hook and loop strap fixed to the first hollow tube.

An adjustable finger splint may include a first hollow tube that may include a first inner surface opposite a first outer surface and defining a first break in the circumference of the first tube; a second hollow tube that may include a second inner surface opposite a second outer surface and defining a second break in the circumference of the second tube; a third hollow tube that may include a third inner surface opposite a third outer surface and defining a third break in the circumference of the third tube; a first connective portion joining the first tube to the second tube; a second connective portion joining the second tube to the third tube; a first hook and loop patch disposed on the first outer surface; a second hook and loop patch disposed on the second outer surface; a third hook and loop patch disposed on the third outer surface; and a hook and loop strap mechanically joined to the first hollow tube and being constructed and arranged to mechanically connect to the first, second, and third hook and loop patches such that the first, second, and third hollow tubes are at rigidly aligned approximately on the same axis of rotation to provide for immobilization of a wearer's finger.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

The features and advantages of the present disclosure will be more fully described in, or rendered obvious by the following detailed description of the preferred embodiments, which are to be considered together with the accompanying drawings wherein like numbers refer to like parts and further, wherein:

FIG. 1 depicts a view from the side of one variation of an adjustable finger splint; and

FIG. 2 depicts an isometric view of one variation of an adjustable finger splint.

### DETAILED DESCRIPTION

Reference will now be made in detail to the present preferred embodiment(s), and examples of which is/are illustrated in the accompanying drawings. Whenever possible, the same reference numerals will be used throughout the drawings to refer to the same or like parts. Any specific details of the embodiments are used for demonstration purposes only, and no unnecessary limitations or inferences are to be understood therefrom.

The following description of the variations is merely illustrative in nature and is in no way intended to limit the scope of the invention, its application, or uses. The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and still are within the scope of the invention.

A number of variations may include a finger splint having two settings wherein the splint may provide for a first setting constructed and arranged to immobilize a user's finger while in the splint. The splint may provide for a second setting wherein the splint may permit a wearer to adjust tension in the stint and move their finger while the splint is still on the user's finger.

A number of variations may include a finger splint including a plurality of tubular components arranged along a shared axis of rotation. The tubular components may be connected by at least one connective portion. The tubular components may also include, individually, a hook and loop patch disposed on an outer portion of the tubular component. A hook and loop strap may be constructed and arranged for providing tension across the tubular components such that a wearers finger may be immobilized within the finger splint. The strap may be temporarily disconnected from the hook and loop patch to provide mobility of a finger within the splint.

A number of variations may include a finger splint that may include a plurality of tubes defining a break in the circumference of each of the tubes. The tubes may include an inner surface opposite an outer surface. Each of the tubes may include a first end opposite a second end. The tubes may define a break in their respective circumference, the break defining a first edge opposite a second edge. The tubes may be aligned along a shared axis of rotation. The tubes may be constructed and arranged to encircle a user's finger and align approximately along the axial length of the finger. The tubes may be made of a malleable material such as a malleable metal and constructed and arranged to circumferentially narrow or compress such that the break in the circumference of each of the tubes may be narrowed or reduced to apply compressive force on a user's finger disposed therein. That is, each of the tubes may be compressed circumstantially such that the first edge and the second edge are drawn towards one another to apply compressive force on a user's finger disposed therein.

The finger splint may include at least one connective portion constructed and arranged to elastically or rigidly connect each of the tubes in the finger splint. The connective position may be mechanically fixed to the inner surface, outer surface, or both, of the tubes such that the tubes are fixed to one another and are aligned approximately along the axial length of a finger. The at least one connective portion may be a plurality of individual connective portions disposed between two adjacent tubes.

The finger splint may include at least one cushion portion disposed on the inner surface of any or all of the tubes. The at least one cushion portion may include a plurality of cushion portions disposed around the inner circumference and inner surface of at least one of the tubes. The at least one cushion portion may be constructed and arranged to cushion a wearer's finger during use and provide comfort as well as compressive force on a user's finger disposed therein.

The finger splint may include at least one hook and loop patch disposed on the outer surfaces of each tube in the finger splint. As a non-limiting example, the at least one hook and loop patch may include a first hook and loop patch disposed on the outer surface of a first tube, a second hook and loop patch disposed on the outer surface of a second tube, and a third hook and loop patch disposed on the outer surface of a third tube. The first, second, and third tubes may be aligned along a shared axis of rotation. Alternatively, the at least one hook and loop patch maybe an elongated patch stretching across a plurality of tubes and connected to each one of the tubes.

The finger splint may include at least one hoop and loop strap fixed to, as a non-limiting example, a first tube and which may be drawn tight over the length of the first tube, the second tube, and third tube, or any plurality of tubes, and which may be constructed and arranged to mechanically engage at least one of the hook and loop patches disposed on the tubes. In this way, tension in the finger splint may be created along the axial length of the finger by attaching the at least one hook and loop strap to at least one hook and loop patch. The finger splint may include a hook and loop strap which may be drawn over and pulled tight against a first hook and loop patch disposed on a first tube, a second hook and loop patch disposed on a second tube, and a third hook and loop patch disposed on a third tube to create tension along the axial length of the finger and render the finger immobile when the finger splint is engaged.

Tension in the finger splint may be removed by disengaging the at least one hook and loop strap from the hook and loop patches. In this way, a user may reduce tension in the finger splint and allow themselves temporary use of the finger inside of the finger splint without removing the finger splint from the finger. In this way, a user may adjust tension in the finger splint while still wearing the finger splint.

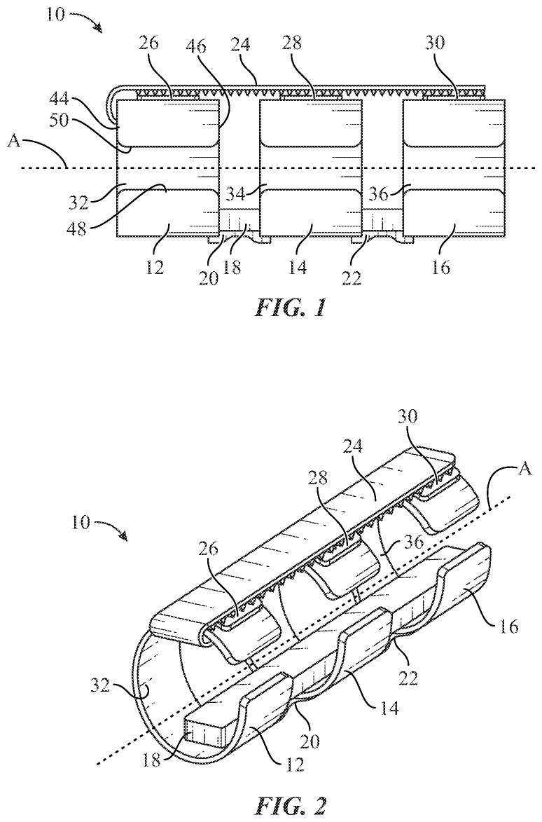

Referring to FIGS. 1 and 2, an adjustable finger splint 10 may include a first tube 12, a second tube 14, and a third tube 16. The first tube 12 may have a first inner face 32 opposite an outer face. The first tube 12 may define a first break 38 in the circumference of the first tube 12. The first tube 12 may be malleable or deformable such that the first tube 12 may be compressed to reduce the size of the first break 38 or otherwise close the first break 38 in order to apply compressive pressure to a wearer's finger disposed therein.

The second tube 14 may have a second inner face 34 opposite an outer face. The second tube 14 may define a second break 40 in the circumference of the second tube 14. The second tube 14 may be malleable or deformable such that the second tube 14 may be compressed to reduce the size of the first break 38 or otherwise close the second break 40 in order to apply compressive pressure to a wearer's finger disposed therein.

The third tube 16 may have a third inner face 36 opposite an outer face. The third tube 16 may define a third break 42 in the circumference of the third tube 16. The third tube 16 maybe malleable or deformable such that the third tube 16 may be compressed to reduce the size of the third break 42 or otherwise close the third break 42 in order to apply compressive pressure to a wearer's finger disposed therein.

The first tube 12 may have a first end 44 opposite a second end 46. The first tube 12 may define a first break 38 in the circumference of the first tube 12. The first break 38 may be defined by a first edge 48 and a second edge 50. Each of the second and third tubes may also have ends, edges, and breaks similar to that of the first tube 12. Each of the tubes may be compressed circumferentially such that the first edge and the second edge are drawn towards one another to apply compressive force on a user's finger disposed therein.

The first tube 12, the second tube 14, and the third tube 16, may be aligned such that they approximately share the same axis of rotation “A”.

At least one cushion 18 may be disposed on at least one of the first tube 12, the second tube 14, or third tube 16. The at least one cushion 18 may include a plurality of cushions wherein each of the first tube 12, the second tube 14, and third tube 16 each have their own respective cushion or cushions disposed thereon. The at least one cushion 18 may include a first cushion disposed on the inner face 32 of the first tube 12 such that portions of or the entirety of the interface 32 is covered by the first cushion and a wearer's finger disposed there in is cushioned circumferentially. Similarly, the at least one cushion 18 may include a second cushion disposed on the second face 34 of the second tube 14 such that portions of or the entirety of the second inner face 34 is covered by the second cushion and a wearers finger disposed therein is cushioned circumferentially. Similarly, the at least one cushion 18 may include a third cushion disposed on the third face 36 of the third tube 16 such that portions of or the entirety of the third inner face 36 is covered by the third cushion and a wearer's finger disposed therein is cushioned circumstantially.

At least one connective portion 20, 22 may be attached to at least one of the first tube 12, the second tube 14, or third tube 16. At least one connected portion 20 may be disposed on the first tube 12 and the second tube 14 such that the first tube 12 and the second tube 14 are connected to one another. At least one connective portion 22 may be disposed on the second tube 14 and the third tube 16 such that the second tube 14 in the third tube 16 are connected to one another. They at least one connected portion may be a plurality of connective portions or single connective portion constructed and arranged to align the first tube 12, the second tube 14, and the third tube 16 along approximately the same axis of rotation A.

A first hook and loop patch 26 may be disposed on an outer face of the first tube 12. A second hook and loop patch 28 may be disposed on an outer face of the second tube 14. A third hook and loop patch 30 may be disposed on an outer face of the third tube 16. At least one hook and loop strap may be connected to at least one of the first tube 12, second tube 14, or third tube 16. The at least one hook and loop strap 24, as an example, may be attached to the first end 44 of the first tube 12. The at least one hook and loop strap 24 may be drawn over and mechanically mate with the first, second, and third hook and loop patches 26, 28, 30 to create tension along the axial length of the finger and render the finger immobile when the finger splint is engaged.

According to variation 1, an adjustable finger splint may include a plurality of hollow tubes that each may include a respective inner surface opposite an outer surface; at least one hook and loop patch disposed on the outer surface; and at least one hook and loop strap constructed and arranged to mechanically connect to the at least one hook and loop patch such that the plurality of hollow tubes are at least semi-rigidly aligned approximately on the same center axis.

Variation 2 may include an adjustable finger splint as set forth in variation 1 that may include at least one cushion disposed on the inner surface.

Variation 3 may include an adjustable finger splint as set forth in any of variations 1 through 2 wherein the plurality of hollow tubes may include a malleable material.

Variation 4 may include an adjustable finger splint as set forth in any of variations 1 through 3 wherein the plurality of hollow tubes may include a first hollow tube, a second hollow tube, and a third hollow tube.

Variation 5 may include an adjustable finger splint as set forth in any of variations 1 through 4 wherein the at least one hook and loop patch may include a first hook and loop patch disposed on a first outer surface of the first hollow tube; a second hook and loop patch disposed on a second outer surface of the second hollow tube; and a third hook and loop patch disposed on a third outer surface of the third hollow tube.

Variation 6 may include an adjustable finger splint as set forth in any of variations 1 through 5 wherein the at least one hook and loop strap is fixed to the first hollow tube.

Variation 7 may include an adjustable finger splint as set forth in any of variations 1 through 6 and further may include at least one connective portion constructed and arranged to attach the first hollow tube to the second hollow tube, and the second hollow tube to third hollow tube such that the first hollow tube, second hollow tube, and third hollow tube share approximately the same axis of rotation.

Variation 8 may include an adjustable finger splint as set forth in any of variations 1 through 7 wherein the at least one connective portion may include a first connective portion attaching the first hollow tube to the second hollow tube and a second connective portion attaching the second hollow tube to third hollow tube.

According to variation 9, an adjustable finger splint may include a first hollow tube that may include a first inner surface opposite a first outer surface and defining a first break in the circumference of the first tube; a second hollow tube that may include a second inner surface opposite a second outer surface and defining a second break in the circumference of the second tube; at least one connective portion mechanically joining the first tube to the second tube; a first hook and loop patch disposed on the first outer surface; a second hook and loop patch disposed on the second outer surface; and a hook and loop strap fixed to the first hollow tube.

Variation 10 may include an adjustable finger splint as set forth in variation 9 that further that may include a third hollow tube that may include a third inner surface opposite a third outer surface and defining a third break in the circumference of the third tube.

Variation 11 may include an adjustable finger splint as set forth in any of variations 9 through 10 wherein the at least one connective portion mechanically joins the second tube to the third tube.

Variation 12 may include an adjustable finger splint as set forth in any of variations 9 through 11 that further may include a third hook and loop patch disposed on the third outer surface.

Variation 13 may include an adjustable finger splint as set forth in any of variations 9 through 12 wherein the at least one connective portion may be mechanically fastened to the first, second, and third outer surfaces such that the first, second, and third tubes are aligned to share approximately the same axis of rotation.

Variation 14 may include an adjustable finger splint as set forth in any of variations 9 through 13 that further may include at least one cushion disposed on the first inner surface, the second inner surface, and the third inner surface.

Variation 16 may include an adjustable finger splint as set forth in any of variations 9 through 14 wherein the first hollow tube, second hollow tube, and third hollow tube may include a malleable material.

Variation 18 may include an adjustable finger splint as set forth in any of variations 9 through 15 wherein the product may be constructed and arranged to immobilize a finger disposed therein along the axial length of the finger.

According to variation 17, an adjustable finger splint may include a first hollow tube that may include a first inner surface opposite a first outer surface and defining a first break in the circumference of the first tube; a second hollow tube that may include a second inner surface opposite a second outer surface and defining a second break in the circumference of the second tube; a third hollow tube that may include a third inner surface opposite a third outer surface and defining a third break in the circumference of the third tube; a first connective portion joining the first tube to the second tube; a second connective portion joining the second tube to the third tube; a first hook and loop patch disposed on the first outer surface; a second hook and loop patch disposed on the second outer surface; a third hook and loop patch disposed on the third outer surface; and a hook and loop strap mechanically joined to the first hollow tube and being constructed and arranged to mechanically connect to the first, second, and third hook and loop patches such that the first, second, and third hollow tubes are at rigidly aligned approximately on the same axis of rotation.

Variation 18 may include an adjustable finger splint as set forth in variation 17 wherein the first hollow tube, second hollow tube, and third hollow tube may include a malleable material.

Variation 19 may include an adjustable finger splint as set forth in any of variations 17 through 18 that further may include at least one cushion disposed on the first inner surface, the second inner surface, and the third inner surface.

Variation 20 may include an adjustable finger splint as set forth in any of variations 17 through 19 wherein the at least one cushion may include a plurality of cushions.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

I claim:

1. An adjustable finger splint, comprising:
a plurality of hollow tubes each comprising a respective inner surface opposite an outer surface;
at least one hook and loop patch disposed on the outer surface; and
at least one hook and loop strap constructed and arranged to mechanically connect to the at least one hook and loop patch such that the plurality of hollow tubes are aligned approximately on the same center axis.

2. The adjustable finger splint as set forth in claim 1, comprising at least one cushion disposed on the inner surface.

3. The adjustable finger splint as set forth in claim 1, wherein the plurality of hollow tubes comprise a malleable material.

4. The adjustable finger splint as set forth in claim 1, wherein the plurality of hollow tubes comprises a first hollow tube, a second hollow tube, and a third hollow tube.

5. The adjustable finger splint as set forth in claim 4, wherein the at least one hook and loop patch comprises:
a first hook and loop patch disposed on a first outer surface of the first hollow tube;
a second hook and loop patch disposed on a second outer surface of the second hollow tube; and
a third hook and loop patch disposed on a third outer surface of the third hollow tube.

6. The adjustable finger splint as set forth in claim 4, wherein the at least one hook and loop strap is fixed to the first hollow tube.

7. The adjustable finger splint as set forth in claim 4, further comprising at least one connective portion constructed and arranged to attach the first hollow tube to the second hollow tube, and the second hollow tube to third hollow tube such that the first hollow tube, second hollow tube, and third hollow tube share approximately the same axis of rotation.

8. The adjustable finger splint as set forth in claim 7, wherein the at least one connective portion comprises a first connective portion attaching the first hollow tube to the second hollow tube and a second connective portion attaching the second hollow tube to third hollow tube.

9. A product, comprising:
a first hollow tube comprising a first inner surface opposite a first outer surface and defining a first break in the circumference of the first tube;
a second hollow tube comprising a second inner surface opposite a second outer surface and defining a second break in the circumference of the second tube;
at least one connective portion mechanically joining the first tube to the second tube;
a first hook and loop patch disposed on the first outer surface;
a second hook and loop patch disposed on the second outer surface; and
a hook and loop strap fixed to the first hollow tube.

10. The product as set forth in claim 9 further comprising a third hollow tube comprising a third inner surface opposite a third outer surface and defining a third break in the circumference of the third tube.

11. The product as set forth in claim 10 wherein the at least one connective portion mechanically joins the second tube to the third tube.

12. The product as set forth in claim 10 further comprising a third hook and loop patch disposed on the third outer surface.

13. The product as set forth in claim 10 wherein the at least one connective portion is mechanically fastened to the first, second, and third outer surfaces such that the first, second, and third tubes are aligned to share approximately the same axis of rotation.

14. The product as set forth in claim 10 further comprising at least one cushion disposed on the first inner surface, the second inner surface, and the third inner surface.

15. The product as set forth in claim 10 wherein the first hollow tube, second hollow tube, and third hollow tube comprise a malleable material.

16. The product as set forth in claim 9 wherein the product is constructed and arranged to immobilize a finger disposed therein along the axial length of the finger.

17. A product, comprising:
a first hollow tube comprising a first inner surface opposite a first outer surface and defining a first break in the circumference of the first tube;
a second hollow tube comprising a second inner surface opposite a second outer surface and defining a second break in the circumference of the second tube;
a third hollow tube comprising a third inner surface opposite a third outer surface and defining a third break in the circumference of the third tube;
a first connective portion joining the first tube to the second tube;
a second connective portion joining the second tube to the third tube;
a first hook and loop patch disposed on the first outer surface;
a second hook and loop patch disposed on the second outer surface;
a third hook and loop patch disposed on the third outer surface; and
a hook and loop strap mechanically joined to the first hollow tube and being constructed and arranged to mechanically connect to the first, second, and third hook and loop patches such that the first, second, and third hollow tubes are rigidly aligned approximately on the same axis of rotation.

18. The product as set forth in claim 17 wherein the first hollow tube, second hollow tube, and third hollow tube comprise a malleable material.

19. The product as set forth in claim 17 further comprising at least one cushion disposed on the first inner surface, the second inner surface, and the third inner surface.

20. The product as set forth in claim 19 wherein the at least one cushion comprises a plurality of cushions.
